(19) United States
(12) Patent Application Publication
(10) Pub. No.: US 2024/0068044 A1
(43) Pub. Date: Feb. 29, 2024

(54) MARKER COMPOSITION FOR PREDICTING PROGNOSIS OF CANCER, METHOD FOR PROGNOSIS OF CANCER AND METHOD FOR PROVIDING INFORMATION FOR DETERMINING STRATEGY OF CANCER TREATMENT

(71) Applicants: Tae Hyun HWANG, Shaker Heights, OH (US); Sunho PARK, Beachwood, OH (US); Jae Ho CHEONG, Seoul (KR); Sung Hak LEE, Seoul (KR); Chung-Kang Sam WANG, Dallas, TX (US); Ryan Matthew POREMBKA, Dallas, TX (US)

(72) Inventors: Tae Hyun HWANG, Shaker Heights, OH (US); Sunho PARK, Beachwood, OH (US); Jae Ho CHEONG, Seoul (KR); Sung Hak LEE, Seoul (KR); Chung-Kang Sam WANG, Dallas, TX (US); Ryan Matthew POREMBKA, Dallas, TX (US)

(21) Appl. No.: 18/280,206

(22) PCT Filed: Dec. 14, 2021
(86) PCT No.: PCT/KR2021/018966

§ 371 Date: Sep. 1, 2023

Related U.S. Application Data
(60) Provisional application No. 63/155,865, filed on Mar. 3, 2021.

(51) Int. Cl. C12Q 1/6886

(52) U.S. Cl. CPC C12Q 1/6886; C12Q 2600/106; C12Q 2600/118; C12Q 2600/158

(57) ABSTRACT

Provided is a marker composition for predicting the prognosis of cancer and a method for predicting the prognosis of cancer using same and, more particularly, to a marker composition for predicting the prognosis of cancer comprising an agent for measuring an expression level of mRNA or protein thereof of at least one gene selected from the group consisting of ESR1, BEST1, ACTA2, HIPK2, IGSF9, ASCC2, JUN, PPP2R5A, SMAD3, CREBBP, EP300, and DDX5, to a method for predicting the prognosis of cancer using same, and to a method for providing information for determining a strategy for treating cancer.

### TECHNICAL FIELD

The present disclosure relates to a marker composition for predicting the prognosis of cancer, a method for predicting the prognosis of cancer using the same, and a method of providing information for determining the treatment direction of cancer and, more particularly, to a marker capable of predicting survival rate, chemo-sensitivity, chemo-resistance, immunotherapy sensitivity, immunotherapy resistance or any combination thereof, a method for predicting the prognosis of cancer using the same, and a method of providing information for determining the treatment direction of cancer, and according to the present disclosure, it is possible to effectively establish a treatment strategy by not only predicting the survival rate of a patient, but also by classifying a patient group that is effective or undesirable to administer a chemical anticancer agent and an immunotherapeutic agent.

### BACKGROUND ART

Many studies have been made to treat cancer, but cancer is an incurable disease that has not yet been conquered. Therapy for diagnosed cancer generally includes surgery, chemotherapy, radiotherapy and the like, but each method has many limitations. In addition, since cancer has a significantly high possibility of recurrence even after treatment, there is a large difference in sensitivity to a chemical anticancer agent and an immuno-anticancer agent depending on the individual, and thus predicting cancer prognosis and sensitivity to chemotherapeutic and immuno-anticancer drugs is essential for determining the treatment direction of cancer patients.

Meanwhile, although many anticancer agents are used as effective therapeutic agents, a newly emerging problem is resistance of cancer cells to anticancer agents. Resistance to an anticancer agent occurs through various mechanisms, such as reducing intracellular accumulation of drugs, activating detoxification or excretion, or modifying target proteins in cells exposed to drugs due to long-term use of an anticancer agent. This process is also deep to the failure of treatment as well as the largest failure elements for cancer treatment. In fact, when chemotherapy is attempted on cancer patients, there are frequent cases where a specific anticancer drug is ineffective and later resistance to other anticancer drugs also illustrates. In the initial treatment, despite the attempt of combination chemotherapy in which several types of anticancer drugs with different mechanisms of action are administered at the same time, it is often observed that there is no therapeutic effect. Therefore, the scope of the anticancer drug that may be used is very limited, which is an important problem in the chemotherapy of cancer.

As a reference to the prognosis prediction at the molecular level currently being used, Microsatellite Instability (MSI), Microsatellite instability (MSI), CpG island methylation phenotype (CIMP), chromosomal instability (CIN), BRAF/KRAS mutations and the like are used, but there is no methodology for predicting the sensitivity of anticancer treatment based on the characteristics of individual patients. Therefore, there is an urgent need to develop a marker capable of accurately predicting the prognosis of cancer patients and at the same time predicting sensitivity to anticancer treatment.

If the prognosis of a patient with respect to the treatment of an anticancer agent after cancer surgery may be predicted, it will be the basis for establishing a treatment strategy suitable for each prognosis. Since 2010, it has been found that adjuvant chemotherapy after standardized gastrectomy increases the survival rate of gastric cancer patients in the case of stage 2 and 3 advanced gastric cancer, and currently, this is a standard treatment. Traditionally, gastric cancer has been classified according to anatomical and pathological phenotype thereof, and according to the TNM stage classification method, chemotherapy is undertaken in cases of stage 2 or higher, but there is no method other than the TNM stage to predict the prognosis according to chemotherapy.

On the other hand, anticancer chemotherapy is essential in the treatment of most cancer patients, which is related to serious adverse effects, and many patients may not get benefits from treatment according to such side effects or may be disadvantageous to the survival rate rather than by side effects. Therefore, when a biomarker for predicting a patient response to chemotherapy is provided, treatment accuracy may be improved, and it is expected to provide a possibility of predicting survival rate and reaction.

### SUMMARY OF INVENTION

### Technical Problem

An aspect of the present disclosure is to provide a marker composition for predicting the prognosis of cancer.

Another aspect of the present disclosure is to provide a method for predicting the prognosis of gastric cancer using the marker composition of the present disclosure.

Another aspect of the present disclosure is to provide a method of providing information for determining a cancer treatment direction using the marker composition of the present disclosure.

### Solution to Problem

According to an aspect of the present disclosure, a marker composition for predicting a prognosis of cancer includes an agent for measuring an expression level of mRNA or protein thereof of at least one gene selected from the group consisting of ESR1, BEST1, ACTA2, HIPK2, IGSF9, ASCC2, JUN, PPP2R5A, SMAD3, CREBBP, EP300 and DDX5.

According to an aspect of the present disclosure, a method of predicting cancer prognosis includes measuring an expression level of mRNA or protein thereof of each gene of the marker composition for predicting a prognosis of cancer of the present disclosure above; and comparing the expression level of mRNA or protein thereof of the measured gene.

According to an aspect of the present disclosure, a method of providing information for determining a treatment direction of cancer includes measuring an expression level of mRNA or protein thereof of at least one gene selected from a I gene group consisting of ESR1, BEST1, ACTA2, HIPK2, IGSF9, ASCC2, JUN, PPP2R5A, SMAD3, CREBBP, EP300 and DDX5, an expression level of mRNA or protein thereof of at least one gene selected from a II gene group consisting of FHL2, PML, BRCA1, WT1, AREG and TP63, and an expression level of mRNA or protein thereof of at least one gene selected from a III gene group consisting of TP53, HSF1, NCOA6IP, PAWR, FAM96A, WTAP, PCNA, GNL3, WRN, SMARCA4, NCOA6, RPA1, MSH6 and PARP1; and by comparing the expression levels of mRNA or protein thereof of the measured gene, classifying as patient group 1 when the expression level of mRNA or protein thereof of the III gene group is relatively high among three gene groups, classifying as patient group 3 when the expression level of mRNA or protein thereof of the II gene group is relatively high, classifying as patient group 4 when the expression level of mRNA or protein thereof of the I gene group is relatively high, and classifying other patients as patient group 2.

### Advantageous Effects of Invention

According to the marker composition for predicting the prognosis of cancer and the method for predicting the prognosis of gastric cancer using the same, and the method of providing information for determining the treatment direction of cancer, it is possible to predict the prognosis of cancer, that is, a survival rate, chemo-sensitivity and resistance, immunotherapy sensitivity and resistance, and the like, thereby preparing a more effective treatment strategy. That is, it is possible to establish an individual patient-customized treatment strategy, such as preventing hypertherapy related to anticancer therapy for patients with good prognosis, actively trying to apply anticancer drugs to a group with poor prognosis but good sensitivity to anticancer treatment, and the like.

### BRIEF DESCRIPTION OF DRAWINGS

FIG. 1 illustrates the results of identifying four molecular subtypes with unsupervised consensus clustering using a 32-gene signature in a Yonsei cohort. That is, the mRNA expression level, especially the expression level of 32 genes, confirmed in tumor tissue of gastric cancer patients (567 patients) of the Yonsei cohort, is a value expressed using z-score, a positive value expressed in each gene means a relatively high mRNA expression level among the target patients, a negative value means a relatively low mRNA expression level, and 0 means the median value.

FIG. 2 illustrates the results of Kaplan-Meier survival analysis of the four molecular subtypes in the Yonsei cohort. A log-rank test was used to examine the statistical significance of differences in overall survival observed between molecular subtypes.

FIG. 3 illustrates the results of Kaplan-Meier survival analysis of molecular subtypes in cohorts of the Asian Cancer Research Group (ACRG) (A of FIG. 3) and Sohn et al. (B of FIG. 3). A log-rank test was used to examine the statistical significance of differences in overall survival observed between molecular subtypes.

FIG. 4 relates to risk scores for predicting 5-year overall survival. A of FIG. 4 illustrates the evaluation of the 5-year overall survival rate by constructing a support vector machine (SVM) with a linear kernel using the expression levels of 32 genes, using the Yonsei cohort as a training set, and illustrates results of application of risk scores to the Asian Cancer Research Group (ACRG), Son et al and Cancer Genome Atlas (TCGA) cohorts. Dotted curves represent 95% confidence section. The rug plot on top of the x-axis represents the risk score for each patient. On the other hand, B of FIG. 4 illustrates a Kaplan-Meier curve for overall survival stratified by risk group, low risk is a risk score below the 25 percentile, moderate risk is a score of the 25 percentile or more and below the 75 percentile, and high risk was defined as a score of the 75 percentile or more.

FIG. 5 illustrates that molecular subtypes of the present disclosure are associated with response to adjuvant 5-fluorouracil (5-FU) and platinum chemotherapy, and illustrates the Kaplan-Meier curves for each group for the overall survival probability of patients treated at Yonsei University. In patients stratified by molecular subtype, patients who underwent surgery without adjuvant chemotherapy and patients who received surgery and adjuvant 5-FU and platinum were compared.

FIG. 6 illustrates whether ACTA2 mRNA and protein expression levels are prognostic for overall survival. A of FIG. 6 illustrates Kaplan-Meier curves for overall survival stratified by Yonsei gastric cancer patient subgroup based on ACTA2 mRNA expression level, absence of ACTA2 mRNA expression is associated with good prognosis, and a subgroup of patients with high levels of ACTA2 mRNA expression has a poor prognosis. B of FIG. 6 illustrates a Kaplan-Meier curve for overall survival stratified by the gastric cancer patient subgroup of Seoul St. Mary Hospital, based on ACTA2 protein expression level, and illustrates that a subgroup of patients with high levels of ACTA2 protein expression is associated with poor overall survival and that a subgroup of patients with low levels of ACTA2 protein expression is associated with good overall survival.

FIG. 7 illustrates a result that a support vector machine (SVM) with a linear kernel learned using 4 molecular subtypes based on 32-gene signatures in the Yonsei cohort is constructed, and then patients who received immunotherapy from Samsung Medical Center (Samsung Medical Center (n=45), Nat Method 2018 September; 24(9):1449-1458. doi: 10.1038/s41591-018-0101-z. Epub 2018 Jul. 16.) are multiclass-classified into four molecular subtypes. The criteria for evaluating the patient's response to immuno-anticancer drugs were classified as complete response (CR), partial response (PR), stable disease (SD), and progressive disease (PD), using the response evaluation criteria in solid tumors (RECIST)k. Among them, patients with CR and PR were classified as an immuno-anticancer drug response group, and SD and PR patients were classified as an immuno-anticancer drug resistant group. FIG. 7 illustrates that the overall response rate (ORR) of immuno-anticancer drug treatment for the I molecular subgroup was 50% (N=10), and the III molecular subgroup had an overall response rate (ORR) of 67% for immuno-anticancer drug treatment. On the other hand, the II molecular subgroup showed an overall response rate (ORR) of immuno-anticancer drug treatment of 7%, and the IV molecular subgroup showed an overall response rate (ORR) of immuno-anticancer drug treatment of 13.3%. Through a chi-square test, it was confirmed that the difference in the overall response rate of immunotherapy between the molecular subgroups was statistically significant (P-value<0.0001).

FIG. 8 illustrates a result of measuring the ACTA2 mRNA expression level through bulk RNA sequencing of tumor tissues of patients who received immuno-anticancer treatment from Samsung Medical Center (n=45), Nat Method 2018 September; 24(9):1449-1458. doi: 10.1038/s41591-018-0101-z. Epub 2018 Jul. 16.), and illustrates that there is a difference in the mRNA expression level of the immune anticancer drug response group (Complete Response; CR and Partial Response; PR) and resistance group (Stable Disease; SD and Progressive Disease; PD) by a box plot. The response group of the immuno-anticancer agent showed a lower level of mRNA expression of the ACTA2 gene than the resistance group. Through statistical methods, it was confirmed that there was a statistically significant difference in the mRNA expression level of the ACTA2 gene between the immuno-anticancer drug response group and the resistance group (P-value=0.00850).

FIG. 9 illustrates that there are the patients of the stomach cancer cohort of The Cancer Genome Atlas (TCGA) into four subgroups through MSI-H and MSS information and the mRNA expression level of ACTA2, that is, 1) MSI-H and ACTA2 high subgroup with high expression of ACTA2 mRNA, 2) ACTA2 low subgroup with MSI-H and low expression level of ACTA2 mRNA, 3) MSS and ACTA2 high subgroup, 4) MSS and ACTA2 low subgroup.

FIG. 10 is a KM plot illustrating that there is a statistically significant difference in overall survival rate between MSI-H or MSS & ACTA2 high or low subgroups of the gastric cancer cohort of TCGA.

### BEST MODE FOR INVENTION

Hereinafter, preferred embodiments of the present disclosure will be described with reference to the accompanying drawings. However, the embodiments of the present disclosure may be modified in various forms, and the scope of the present disclosure is not limited to the embodiments described below.

The present inventors have discovered that the prognosis of cancer, that is, the suitability of survival rate and anticancer therapy application, may be predicted on the basis of the expression levels of 32 genes included in the changed pathway specific to gastric cancer. At this time, the survival rate includes an overall survival rate, for example, an overall survival rate of 5 years. The 32-gene assay of the present disclosure may have the potential to improve the accuracy of cancer treatment.

In the present specification, the term “expression of a gene” is intended to include the expression level of mRNA of a gene or protein thereof.

In the present disclosure, “prognosis” refers to predicting various states of a patient according to cancer, such as the possibility of a complete cure of cancer, possibility of recurrence after treatment, possibility of survival of a patient, and the like after cancer is diagnosed, and in the present disclosure, for example, survival rate, chemo-sensitivity, chemo-resistance, immunotherapy sensitivity, or any combination thereof as the treatment prognosis of anticancer therapy. For purposes of the present disclosure, prognosis may refer to prognosis for survival and prognosis for treatment after diagnosis of cancer. When the marker provided by the present disclosure is used, survival prognosis of cancer patients and prognosis of anticancer therapy treatment may be more easily predicted, and thus, it may be used to classify patients of high risk groups or to determine whether to use additional necessary treatment methods, thereby contributing to increasing survival rates after cancer development.

In addition, the term “prediction” is related to whether or not a patient survives or the possibility thereof after treatment of a patient in a preferred or non-preferred response to the therapy. The marker compositions of the present disclosure may be clinically used to make therapeutic decisions by selecting the most appropriate treatment scheme for cancer onset patients. In addition, the prediction method of the present disclosure may be used to check whether a patient is preferred for a treatment prescription, for example, or to predict whether the patient may survive the long-term survival of the patient after the treatment prescription.

The term “anticancer therapy” used in the present disclosure is intended to include a treatment using a (chemical) anticancer agent and/or an immunotherapeutic agent.

More specifically, the marker composition for predicting the prognosis of cancer of the present disclosure includes an agent for measuring the expression level of mRNA or protein thereof of at least one gene selected from the group consisting of ESR1, BEST1, ACTA2, HIPK2, IGSF9, ASCC2, JUN, PPP2R5A, SMAD3, CREBBP, EP300 [symbol] DDX5.

More preferably, the composition includes an agent for measuring the expression level of the mRNA of the ACTA2 gene or protein thereof, and may include an agent for measuring the expression level of mRNA or protein thereof of at least one, or at least two, or the total genes, selected from the group consisting of the ACTA2 gene and ESR1, BEST1, HIPK2, IGSF9, ASCC2, JUN, PPP2R5A, SMAD3, CREBBP, EP300 and DDX5.

For example, it may include an agent for measuring the expression level of mRNA or protein thereof of at least one gene selected from the group consisting of BEST1, ACTA2, ESR1, CREBBP and EP300.

Furthermore, the marker composition for predicting the prognosis of cancer of the present disclosure may further include an agent for measuring the expression level of mRNA or protein thereof of at least one, or at least two, or of the total genes, selected from the group consisting of FHL2, PML, BRCA1, WT1, AREG and TP63.

In addition, the marker composition for predicting the prognosis of cancer according to the present disclosure may further include an agent for measuring the expression level of mRNA or protein thereof of at least one, or at least two, or of the total genes, selected from the group consisting of TP53, HSF1, NCOA6IP, PAWR, FAM96A, WTAP, PCNA, GNL3, WRN, SMARCA4, NCOA6, RPA1, MSH6 [symbol] PARP1.

In the present disclosure, for the sake of convenience, a gene group consisting of ESR1, BEST1, ACTA2, HIPK2, IGSF9, ASCC2, JUN, PPP2R5A, SMAD3, CREBBP, EP300 and DDX5 may be referred to as an I gene group; a gene group consisting of FHL2, PML, BRCA1, WT1, AREG and TP63 may be referred to as a II gene group; and also a gene group consisting of TP53, HSF1, NCOA6IP, PAWR, FAM96A, WTAP, PCNA, GNL3, WRN, SMARCA4, NCOA6, RPA1, MSH6 and PARP1 may be referred to as a III gene group.

The cancer of which the prognosis may be predicted using the marker composition of the present disclosure may be selected from the group consisting of gastric cancer, bladder cancer, kidney cancer, brain cancer, uterine cancer, skin cancer, pancreatic cancer, lung cancer, colorectal cancer, liver cancer, and breast cancer, and is preferably gastric cancer.

In the present specification, the term “measurement of the expression level of mRNA” refers to measuring the amount of mRNA in a process of confirming the mRNA expression level of genes in a biological sample. Analysis methods therefor are reverse transcription polymerase reaction (RT-PCR), competitive reverse transcription polymerase reaction (Competitive RT-PCR), real-time reverse transcription polymerase reaction (Real-time RT-PCR), RNase protection assay (RPA), Northern blotting, DNA chip, etc., but are not limited thereto.

In the composition according to the present disclosure, the agent for measuring the mRNA expression level of a gene includes a primer, a probe, or an antisense nucleotide that specifically binds to mRNA of each gene. Since the information of each gene according to the present disclosure is known from GenBank, UniProt, etc., a person skilled in the art can easily design primers, probes, or antisense nucleotides that specifically bind to the mRNA of each gene based on this information.

The term “primer” in the present disclosure means a single-stranded oligonucleotide that may act as the starting point of template-directed DNA synthesis under suitable conditions (i.e., four different nucleoside triphosphates and polymerase) in a suitable temperature and suitable buffer. Suitable lengths of primers may vary depending on the use of various elements, such as temperature and primer. In addition, the sequence of the primer does not need to have a sequence that is completely complementary to some sequences of the template, and it is sufficient to have sufficient complementarity within a range capable of hybridizing with the template and performing the intrinsic action of the primer. Therefore, the primer in the present disclosure does not need to have a sequence which is perfectly complementary to the nucleotide sequence of each gene which is a template, and it is sufficient if the primer has sufficient complementarity within a range capable of performing a primer action by being hybridized with the gene sequence. The primers include primer pairs in forward and reverse directions, but are preferably primer pairs that provide analysis results with specificity and sensitivity. The nucleic acid sequence of the primer does not match the non-target sequence present in the sample, and thus when only the target gene sequence containing the complementary primer binding site is amplified and non-specific amplification is not caused, high specificity may be imparted.

The term “amplification reaction” refers to a reaction of amplifying nucleic acid molecules, and the amplification reactions of these genes are well known in the art, and may include, for example, polymerase chain reaction (PCR), reverse transcription polymerase chain reaction (RT-PCR), ligase chain reaction (LCR′), electron mediated amplification (TMA), nucleic acid base sequence substrate amplification (NASBA), and the like.

In the present disclosure, the term “probe” refers to a linear oligomer of a natural or modified monomer or linkage, includes deoxyribonucleotides and ribonucleotides, may specifically hybridize to a target nucleotide sequence, and is naturally present or artificially synthesized. The probe according to the present disclosure may be a single chain, preferably an oligodeoxyribonucleotide. The probe of the present disclosure may include natural dNMPs (i.e., dAMP, dGMP, dCMP and dTMP), nucleotide analogues or derivatives. In addition, the probe of the present disclosure may also include ribonucleotides.

In addition, in the present disclosure, the expression level of the protein preferably indicates a polypeptide generated through a translation process from mRNA in which each gene is expressed, and a material capable of measuring the level of each protein may include an antibody, such as a polyclonal antibody, a monoclonal antibody, a recombinant antibody and the like, which may specifically bind to each protein.

The marker composition for predicting the prognosis of cancer of the present disclosure may further include a pharmaceutically acceptable carrier. The pharmaceutically acceptable carrier includes a carrier and vehicle commonly used in the medical field, and specifically includes an ion exchange resin, alumina, aluminum stearate, lecithin, serum protein (e.g., human serum albumin), a buffer material (e.g., various phosphates, glycine, sorbic acid, potassium sorbates, partial glyceride mixtures of saturated vegetable fatty acids), water, salts or electrolytes (e.g., protamine sulfate, disodium hydrogen phosphate, potassium hydrogen phosphate, sodium chloride and zinc salts), glial silica, magnesium trisilicate, polyvinylpyrrolidone, cellulose based substrate, polyethylene glycol, sodium carboxymethylcellulose, polyarylate, wax, polyethylene glycol or wool, and the like, but is not limited thereto.

In addition to the above components, a lubricant, a wetting agent, an emulsifier, a suspending agent, a preservative, or the like may be further included.

According to another aspect of the present disclosure, there is provided a method for predicting the prognosis of cancer using the marker composition of the present disclosure.

More specifically, the method for predicting the prognosis of cancer according to the present disclosure includes the operations of: measuring the expression level of mRNA of each gene or protein thereof of the marker composition for predicting the prognosis of cancer; and comparing the expression level of mRNA of the measured gene or the expression level of the protein thereof.

The comparison may be performed by relatively comparing the expression levels of mRNA of the measured gene, or the expression level of the protein thereof, and in this case, various methods known in the art may be used to compare the expression level of mRNA or protein thereof, and in addition, the comparison may be processed using a known data analysis method. For example, methods such as Nearest Neighbor Classifier, Partial-Least Squares, SVM, AdaBoost, clustering-based classification, or the like may be used. Also, to confirm significance, various statistical processing methods may be used. In one embodiment, a logistic regression analysis method may be used as a statistical processing method.

The method for predicting the prognosis of cancer, according to the present disclosure, may further include determining that the prognosis of chemotherapy will be poor and/or that the prognosis of immunocancer treatment is poor, when at least one gene selected from the first gene group consisting of ESR1, BEST1, ACTA2, HIPK2, IGSF9, ASCC2, JUN, PPP2R5A, SMAD3, CREBBP, EP300 and DDX5, for example, when the expression level of mRNA or protein thereof of at least one of ACTA2, ESR1, BEST1, HIPK2, ASCC2, JUN, EP300, CREBBP and DDX5 is relatively high, preferably, when the expression level of ACTA2 mRNA or protein thereof is relatively high.

In addition, using the marker composition of the III gene group, it may further include determining that the prognosis of chemotherapy is poor and the prognosis of immunotherapy is good, when the expression level of mRNA or protein thereof of at least one gene selected from the group consisting of TP53, HSF1, NCOA6IP, PAWR, FAM96A, WTAP, PCNA, GNL3, WRN, SMARCA4, NCOA6, RPA1, MSH6 and PARP1, for example, of at least one of TP53, HSF1, NCOA61P, PAWR, FAM96A, WTAP, PCNA, GLN3, WRN, WRN, SMARCA4, NCOA6, RPA1, MSH6 and PARP1 is relatively high, more preferably, at the same time, when the expression level of ACTA2 mRNA or protein thereof is relatively low.

Furthermore, when the expression level of mRNA or protein thereof of at least one gene selected from the group consisting of FHL2, PML, BRCA1, WT1, AREG and TP63, for example, of at least one of FML2, PML, BRCA1, WT1, AREG, and TP63 is relatively high, and more preferably, simultaneously therewith, when the expression level of ACTA2 mRNA or protein thereof is relatively low, operation of determining that the prognosis of the chemotherapy and/or the immunotherapy will be good may be further included.

In this case, the prognosis may be survival rate, chemo-sensitivity, chemo-resistance, immunotherapy sensitivity, immunotherapy resistance, or any combination thereof.

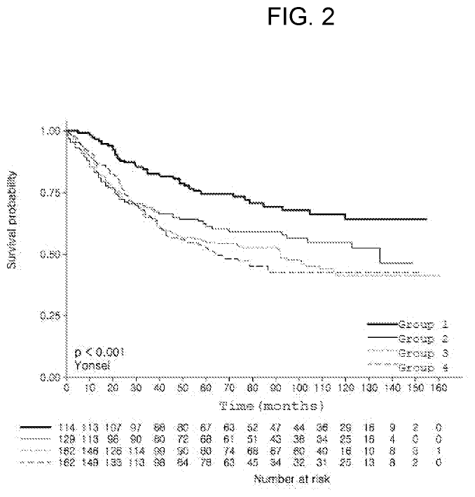

In addition, referring to FIG. 2, significant differences in the overall survival rate were confirmed between groups, Group 1 patients of the III gene group with high expression levels showed the best results, whereas Group 4 patients of the I gene group with high expression levels showed the worst result.

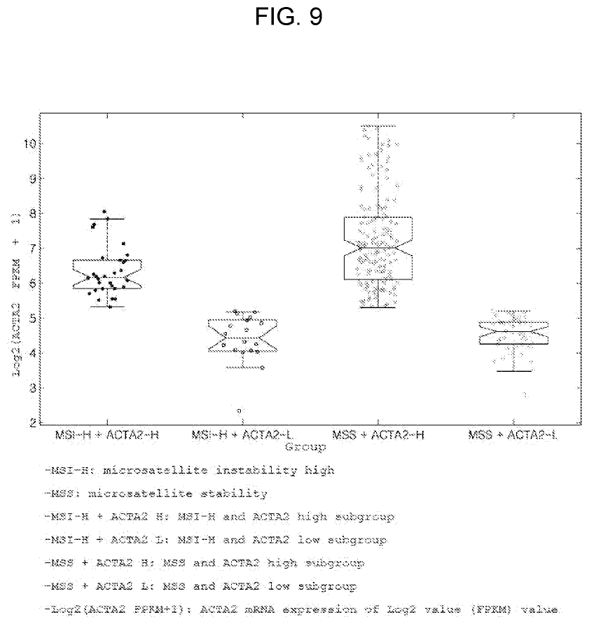

In the present disclosure, it is relatively determined whether the expression level of mRNA of a gene or a protein thereof is high, by comparing the expression level of mRNA of the measured gene or the protein thereof, and for example, it may be determined that the expression level is high based on the total average expression level of mRNA or protein thereof of the measured gene when exceeding the same. For example, by converting the mRNA expression level into a z-score and illustrating a heat map, it may be determined that the expression level of the gene corresponding to the positive region is high. For example, in the case of ACTA2, when the mRNA expression level using bulk mRNA sequencing is relatively equal to or greater than, by comparison to the expression level of mRNA or protein thereof of the gene whose log 2 (Fragments Per Kilobase of transcript per Million mapped reads (FPKM)+1) is measured, and/or in the case of immunohistochemistry, when the score calculated by respectively multiplying the staining intensity and the staining area score is greater than 3, it may be determined that the ACTA2 expression level is high. In addition, when the Log 2 (FPKM+1) value is relatively small compared to the expression level of mRNA or protein thereof of the measured gene, and/or in the case of immunohistochemical staining, when the score calculated by respectively multiplying the staining intensity and the staining area score is equal to or less than 3, ACTA2 expression level may be classified as low. For example, referring to FIG. 9, when the Log 2(FPKM+1) value is 5 or more, the ACTA2 expression level is high, and when the value is less than 5, it may be regarded that the expression level is low. Other genes may also be classified as high or low in the expression level of the gene in the same or similar manner.

That is, it can be seen that since the marker composition of the present disclosure may confirm the association with the mortality risk independently, it can be a prognostic criterion independently of clinical and pathological variables known in the art.

According to still another aspect of the present disclosure, provided is a method of providing information for determining a cancer treatment direction.

More specifically, the method of providing information for determining the treatment direction of cancer according to the present disclosure includes, measuring an expression level of mRNA or protein thereof of at least one gene selected from a I gene group consisting of ESR1, BEST1, ACTA2, HIPK2, IGSF9, ASCC2, JUN, PPP2R5A, SMAD3, CREBBP, EP300 and DDX5, an expression level of mRNA or protein thereof of at least one gene selected from the II gene group consisting of FHL2, PML, BRCA1, WT1, AREG and TP63, and an expression level of mRNA or protein thereof of at least one gene selected from the III gene group consisting of TP53, HSF1, NCOA6IP, PAWR, FAM96A, WTAP, PCNA, GNL3, WRN, SMARCA4, NCOA6, RPA1, MSH6 and PARP1; and by comparing the expression levels of mRNA or protein thereof of the measured gene, classifying as patient group 1 when the expression level of mRNA or protein thereof of the III gene group is relatively high among three gene groups, classifying as patient group 3 when the expression level of the mRNA or protein thereof of the II gene group is relatively high, classifying as patient group 4 when the expression level of mRNA or protein thereof of the I gene group is relatively high, and classifying other patients as group 2.

The patient group 2 may be a patient in which the expression level of mRNA of the I to III gene groups is not distinguished between the I to III gene groups, that is, refers to a case in which the expression level of gene in a specific gene group does not tend to increase over the I to III gene groups.

The method of providing information for determining the treatment direction of cancer according to the present disclosure may further include at least one operation of: predicting that the anticancer therapy using the chemical anticancer agent is unsuitable for the patient group 1; predicting that the anticancer therapy using the chemical anticancer agent is suitable for the patient group 3; and predicting that the anticancer therapy using the chemical anticancer agent is unsuitable for the patient group 4.

In this case, the anticancer agent may be a complex anticancer therapy in which at least one chemical anticancer agent selected from fluorouracil (5-FU), bleomycin, and epirubicin is combined based on platinum, and is preferably a complex anticancer therapy of platinum or platinum and fluorouracil (5-FU).

In the present disclosure, Group 3 patients showed improved survival rates in relation to anticancer therapy using 5-FU butter and platinum-based chemical anticancer agents, and in the case of a group 2 patient, it was confirmed that improved survival rates showed in relation to therapy using 5-FU-based single chemical anticancer agent.

Furthermore, interestingly, Group 3 patients showed good response to both 5-FU and platinum doublet chemotherapy and anti-PD-1 therapy, and thus a clinical trial of a combination of a chemical anticancer agent and an immune anticancer agent may be considered in the patient population.

On the other hand, although the group 1 patient exhibited the best prognosis, it was confirmed that the prognosis was deteriorated when anticancer therapy using a chemical anticancer agent, for example, 5-FU SCF and platinum treatment therapy is applied. Accordingly, a strategy for excluding anticancer therapy using a chemical anticancer agent may be considered for the group 1 patient.

Furthermore, the method of providing information for determining a treatment direction of cancer according to the present disclosure may include at least one operation of: predicting that at least one patient group of patient group 1 and patient group 3 is suitable for immunotherapy using an immunotherapeutic agent; and predicting that at least one patient group of patient group 2 and patient group 4 is unsuitable for immunotherapy using an immunotherapeutic agent.

In this case, the immunotherapeutic agent may be at least one immunotherapeutic agent selected from anti-PD1 inhibitor, an anti-CTLA4 immunotherapeutic agent, and an anti-PDL1 immunotherapeutic agent.

Furthermore, the method of providing information for determining the treatment direction of cancer of the present disclosure may further include an operation of diagnosing microsatellite instability (MSI) to determine the treatment direction of cancer.

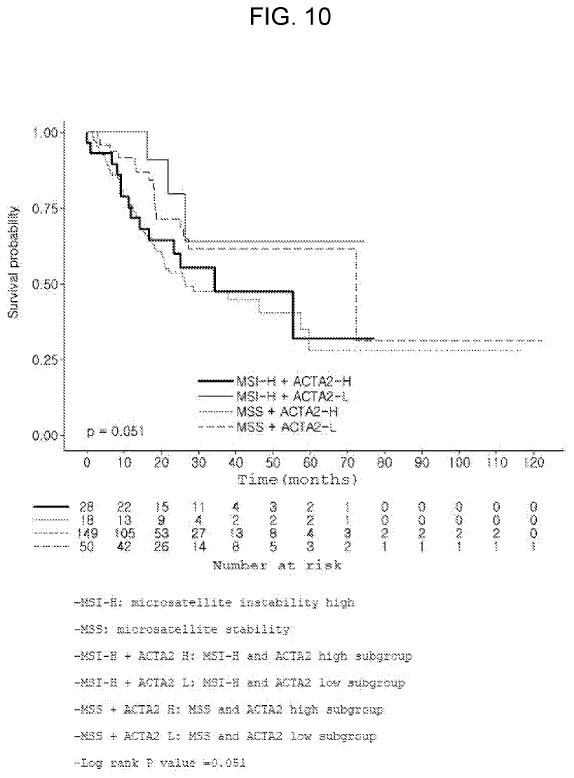

For example, even when microsatellite instability (MSI) is diagnosed and confirmed as a high-frequency microsatellite instability high (MSI-H) patient, as can be seen in FIG. 10, it can be confirmed that the biomarker of the present invention, for example, the survival possibility is significantly different between high and low expression of at least one of the I gene group, preferably the ACTA2 gene.

Therefore, by combining the marker composition for predicting the prognosis of cancer of the present disclosure together with microsatellite instability (MSI) diagnosis widely used in the art, it is expected that a patient may be classified into more detailed groups, which have not been conventionally classified, and prognosis may be predicted to determine the most effective treatment direction suitable for a patient. According to the marker composition for predicting the prognosis of cancer and the method for predicting the prognosis of gastric cancer using the same, and the method of providing information for determining the treatment direction of cancer according to the present disclosure, since cancer prognosis, immunotherapy sensitivity and/or chemo-sensitivity may be predicted, a more effective treatment strategy may be prepared.

That is, it is possible to establish an individual patient-customized treatment strategy such as, for example, preventing anticancer therapy-related hypertherapy for patients with good prognosis, and actively trying to apply anticancer therapy to a group with poor prognosis but good sensitivity to anticancer therapy, and the like.

Hereinafter, the present disclosure will be described in more detail with reference to detailed examples. The following examples are merely examples for helping the understanding of the present disclosure, and the scope of the present disclosure is not limited thereto.

### MODE FOR INVENTION

### Examples

1. Identification of Gene Signature and Molecular Subtype

To identify biomarkers for predicting prognosis in gastric cancer, the somatic mutation profiles of 6,681 patients from 19 different cancer types published by The Cancer Genome Atlas (TCGA) was input to NTriPath and the pathway specifically altered in gastric cancer was identified.

To investigate the prognosis prediction related utility of these pathways, the present inventors generated microarray-based mRNA expression profiles from pre-treated tumor samples from 567 patients who underwent resection at Yonsei University. The 89% of the patient had a stage II or III disease and a median duration of the follow-up period was 61 months.

It was confirmed that the gastric cancer-specific pathway useful for prognosis prediction contains 32 genes of Table 1 below, including TP53, BRCA1, MSH6, PARP1, and ACTA2, integrated in DNA damage response, TGF-β signaling, and cell proliferation pathways.

TABLE 1

| No. | Gene |
|---|---|
| 1 | ACTA2 |
| 2 | AREG |
| 3 | ASCC2 |
| 4 | BEST1 |
| 5 | BRCA1 |
| 6 | CREBBP |
| 7 | DDX5 |
| 8 | EP300 |
| 9 | ESR1 |
| 10 | FAM96A |
| 11 | FHL2 |
| 12 | GNL3 |
| 13 | HIPK2 |
| 14 | HSF1 |
| 15 | IGSF9 |
| 16 | JUN |
| 17 | MSH6 |
| 18 | NCOA6 |
| 19 | NCOA6IP |
| 20 | PARP1 |
| 21 | PAWR |
| 22 | PCNA |
| 23 | PML |
| 24 | PPP2R5A |
| 25 | RPA1 |
| 26 | SMAD3 |
| 27 | SMARCA4 |
| 28 | TP53 |
| 29 | TP63 |
| 30 | WRN |
| 31 | WT1 |
| 32 | WTAP |

Among these genes, FHL2, PML, BRCA1, WT1, AREG and TP63 are genes in apoptosis signaling and cell proliferation paths, and are referred to as I gene groups; ESR1, BEST1, ACTA2, HIPK2, IGSF9, ASCC2, JUN, PPP2R5A, SMAD3, CREBBP, EP300 and DDX5 are genes found in TGF-β, SMAD and estrogen receptor signaling and mesenchymal morphogenesis pathways, and are referred to as the II gene group; and TP53, HSF1, NCOA6IP, PAWR, FAM96A, WTAP, PCNA, GNL3, WRN, SMARCA4, NCOA6, RPA1, MSH6 and PARP1 are genes involved in cell cycle, DNA damage response and recovery, and mismatch recovery, and are referred to as the III gene group.

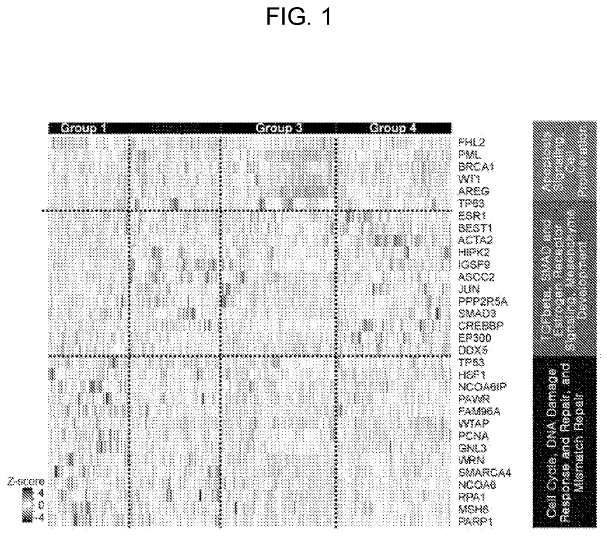

The present inventors have performed consensus clustering based on the expression levels of the 32 genes, and found four distinct molecular subtypes of Groups 1 to 4 based on the consensus cumulative distribution function (CDF) plot and delta area plot as well as manual examination of the consensus matrix. A molecular subtype was found (FIG. 1).

Tumors from Group 1 patients have expressed genes associated with cell cycle, DNA damage response and recovery, and mismatch recovery, and cancer from Group 4 patients overexpressed genes found in TGF-β, SMAD and estrogen receptor signaling and mesenchymal form generation pathways. Tumors from Group 3 patients over-expressed genes in apoptosis signaling and cell proliferation paths. The tumors from Group 2 did not show a unique pattern of overexpressed genes. In this case, whether or not overexpression is determined by relatively comparing the expression levels of the 32 genes.

In one variation analysis, the molecular subtype was significantly correlated with the difference in age (p=0.003), operation (p=0.021), Lauren type (p<0.001), and perineuronal attack (P<0.001). Finally, significant differences were observed between groups at the overall survival rate. Group 1 patients showed the best results, and it can be confirmed that the survival probability of group 1 patients reaches about 70%, compared to the survival probability after 150 months of patients in other groups, all less than 50%, while group 4 patients showed the worst outcome, and the median overall survival rate was 65 months (FIG. 2; P<0.001).

The multivariable Cox proportional-risk analysis using significant variables for the single variable analysis showed that the molecular subtypes of the present disclosure, such as age, operation, etc. are independently associated with a mortality risk (Table 2). That is, this indicates that the 32-gene signature of the present disclosure may be a standard that acts independently of known important clinical and pathological variables.

TABLE 2
Multivariate Analysis of Yonsei
Gastric Cancer Molecular Subtypes

| | | |
|---|---|---|
| Column | Hazard Ratio, 95% CI | P-val |

Age

| | | |
|---|---|---|
| Age = <60 | 1.00 | reference |
| Age > 60 | 1.94782 (1.51438, 2.50531) | 0.000 |

Stage

| | | |
|---|---|---|
| I | 1.00000 | reference |
| II | 1.86018 (0.65875, 5.25278) | 0.241 |
| III | 3.57560 (1.31076, 9.75385) | 0.013 |
| IV | 18.19782 (6.07559, 54.50672) | 0.000 |

Tumor Location

| | | |
|---|---|---|
| Antrum | 1.00000 | reference |
| Body | 1.02265 (0.78048, 1.33995) | 0.871 |
| Cardia | 0.90207 (0.56102, 1.45044) | 0.671 |
| Whole | 1.48787 (0.59556, 3.71715) | 0.395 |

Lauren Type

| | | |
|---|---|---|
| Diffuse | 1.00000 | reference |
| Intestinal | 0.89238 (0.65022, 1.22473) | 0.481 |
| Mixed | 0.70872 (0.35366, 1.42028) | 0.332 |
| Other | 1.19781 (0.85501, 1.67804) | 0.294 |

Perineural Invasion Positive

| | | |
|---|---|---|
| Negative | 1.00000 | reference |
| Positive | 1.12046 (0.81042, 1.54911) | 0.491 |

Molecular Subtype Group

| | | |
|---|---|---|
| Group 1 | 1.00000 | reference |
| Group 2 | 1.96829 (1.31362, 2.94921) | 0.001 |
| Group 3 | 1.72567 (1.12222, 2.65360) | 0.013 |
| Group 4 | 2.18175 (1.43763, 3.31103) | 0.000 |

2. Demonstration of 32-Gene Prognostic Signature

To investigate the robustness and reproducibility of the 32-gene prognostic signature, as an independent data set, the present inventors have analyzed the gene expression profiles of gastric cancer patients published by the Asian Cancer Research Group (ACRG; n=300; Gene Expression Omnibus: GSE62254) and by Sohn et al.) (n=267; gene expression omnibus: GSE13861 and GSE26942).

Four molecular subtypes were identified according to the present disclosure again with unsupervised consensus clustering using the 32-gene signature. Subtypes of ACRG cohorts were correlated with age (p=0.001), gender (p=0.016), operation (p=0.001), tumor location (p=0.004), Lauren type (p<0.001), neuron peripheral attack (p<0.001), EBV status (p=0.03) and ACRG molecular subtype classification (P<0.001; (Table S5)). The sub-types in Sohn et al's cohort were significantly correlated with differences in sex (p=0.032), Lauren type (p=0.04), TCGA molecule grouping (P<0.001; table S6).

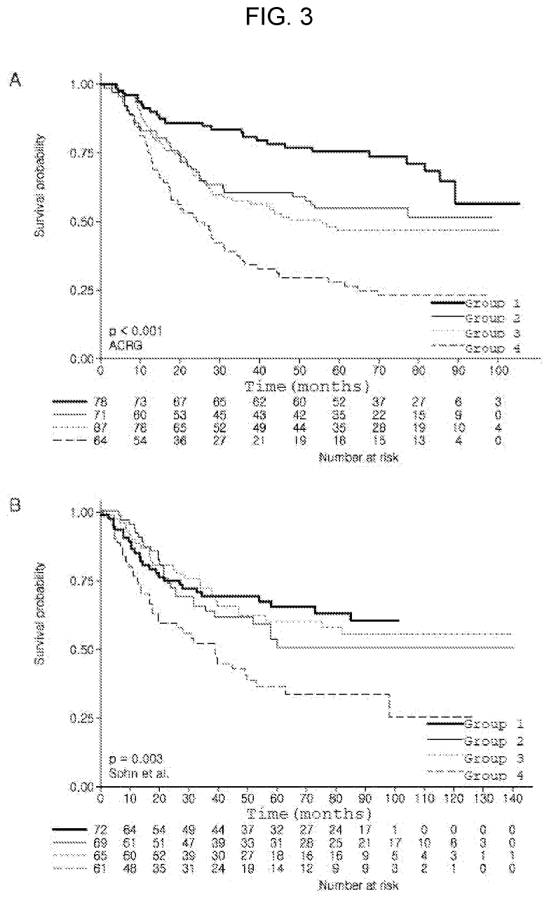

Meanwhile, in both cohorts, the molecular subtype of the present disclosure was confirmed to be significantly associated with the survival rate (FIGS. 3A and 3B). Multivariable Cox proportional-risk analysis of cancer operations, Lauren types, tumor locations and molecular subtypes associated with mortality risk in both cohorts of ACRG, Sohn et al. and like showed that molecular subtypes were significantly associated particularly with the survival rate between Group 1 and Group 4, among others. As a result of the analysis, it was confirmed that the 32-gene signature may be an important prognostic biomarker.

3. Machine Learning to Identify Risk Scores for Predicting Overall Survival Rate in Five Years

Using the Yonsei cohort as a training set, the present inventors constructed a support vector machine (SVM) with a linear kernel that uses the 32 gene expression levels to evaluate the overall survival rate in five years.

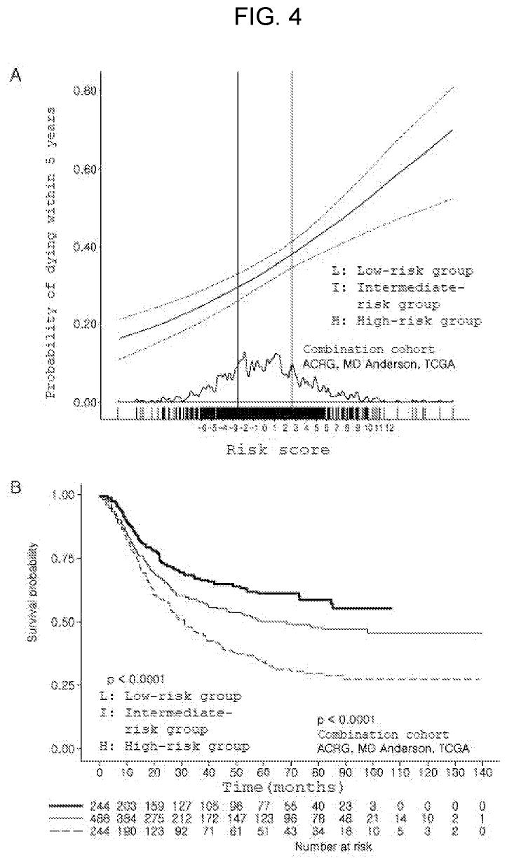

Group 1 with the best prognosis was administered a voice label and Group 4 with the worst prognosis was administered positive labels. The inventors of the present disclosure tested an SVM model using data published by ACRG, Sohn et al. and cancer genomic atlas, and confirmed that the risk score as a continuous variable predicted the 5-year overall survival rate (FIG. 4).

The present inventors have divided cohorts into quartiles on the basis of a risk score. Patients in the lower quartile were classified as a low risk, patients within the range between the quartiles were classified as intermediate risk, and patients in the upper quartile were classified as high risk. The 5-year overall survival rates for the low-, intermediate-, and high-risk groups were 61% (95% CI, 55%-69′), 50% (45′-56j), and 35% (28%-42%), respectively (FIG. 3B; P<0.0001). Importantly, the risk score was prognostic regardless of clinical and pathological characteristics known to be associated with poor results over all datasets (Table 3 and 513-15). These results demonstrated that the risk score derived from machine learning on the basis of the 32-gene signature predicts 5-year survival rate in gastric cancer patients.

4. Molecular Subtype Prediction Reactions for Systemic Therapy

It has been examined whether the molecular subtype of the present disclosure may predict the response to the systemic therapy. The Yonsei cohort included patients treated prior to establishment of adjuvant chemotherapy as standard of care. Thus, patients who have been treated with one of the following three assisted chemical therapy methods were able to compare patients who have undergone surgery only:

5-Fluorouracil (5-FU) Alone Therapy

5-FU and Platinum Doublet

Treatment of Systemic Therapy of Another Class in Addition to 5-FU

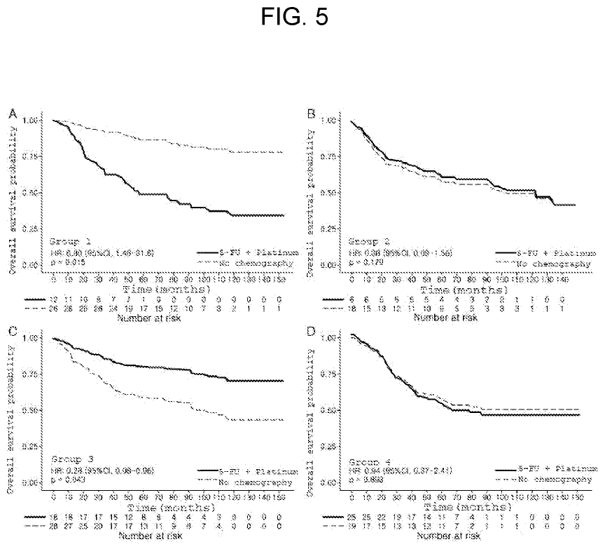

The inventors of the present disclosure performed a multivariable Cox proportional analysis of the overall survival rate, assisted chemical therapy, cancer operation, age, lymphovascular attack and perineuronal attack as covariates within each genetic group. The present inventors found that patients treated with 5-FU and platinum in Group 3 exhibited significantly better overall survival rates, compared to Group 3 patients not subjected to assisted chemical therapy (hazard ratio (HR), 0.28(95% CI, 0.08-0.96), P=0.043). In contrast, however, patients in Group 1 treated with 5-FU and platinum showed a poorer survival rate than Group 1 patients not subjected to assisted therapy (HR, 6.80(95% CI, 1.46-31.6), P=0.015), (FIG. 5). On the other hand, Group 2 patients showed improved survival rates associated with 5-FU monotherapy (HR, 0.37 (95% CI, 0.14-0.99), and addition of other agents was not correlated with improved outcome. These data suggest that the molecular subtype of the present disclosure is a predictive biomarker for assisted therapy.

Then, it was examined whether the sub-type of the present disclosure may predict a response to an immune anticancer agent, for example, an immune checkpoint inhibitor, and as a result of analyzing the cohorts of patients with refractory, metastatic and/or recurrent gastric cancer, treated with anti-PD1 inhibitor, anti-CTLA4 immuno-anticancer, or anti-PDL1 immuno-anticancer as immunotherapy, it was confirmed that the molecular subtype of the present disclosure was associated with immunotherapy reaction and resistance (FIG. 7).

Looking at the results of recent randomized control trials, the overall response rate (ORR) of patients with refractory, metastatic and or recurrent gastric cancer, treated with immunotherapy was less than 20%(12% in KEYNOTE-059 (Fuchs et al, JAMA ONC, 2018), 16% in KEYNOTE-061 (Shitara et al, Lancet, 2018), 11% in ATTRACTION-2 (Kang et al, Lancet, 2017)).

On the other hand, referring to the results of FIG. 7, in the case of the classification of patient groups using the molecular subtype of the present disclosure, that is, the I to III gene groups, and the method for predicting the prognosis of cancer based thereon, the patient group I showed an overall reaction rate (ORR) of 50% (N=10), and the patient group III showed an overall reaction rate (ORR) of 67% of the immunotherapeutic agent treatment. Therefore, according to the prognosis prediction method of the present disclosure, it can be seen that it is remarkably effective, as compared to a method for selecting a patient responding to a currently used immunotherapeutic agent, and according to the present disclosure, it was confirmed that the reaction to the immune checkpoint inhibitor may also be predicted.

TABLE 3
Hazard Ratio (HR) for specific chemotherapy and no-chemotherapy
treatment in each patient group of the present disclosure,
obtained by multivariate Cox proportional analysis

| | | Hazard Ratio |
|---|---|---|
| Patient Group | P-value | (95% CI)^{a} |
| Patient Group 1 | | |
| 5-FU alone VS no-chemotherapy | 0.226 | 2.25 (0.61, 8.35) |
| 5-FU + Platinum VS no-chemotherapy | 0.015 | 6.80 (1.46, 31.63) |
| 5-FU + others VS no-chemotherapy | 0.429 | 2.00 (0.36, 11.08) |
| Patient Group 2 | | |
| 5-FU alone VS no-chemotherapy | 0.049 | 0.37 (0.14, 0.99) |
| 5-FU + Platinum VS no-chemotherapy | 0.179 | 0.38 (0.09, 1.56) |
| 5-FU + others VS no-chemotherapy | 0.462 | 0.69 (0.26, 1.84) |
| Patient Group 3 | | |
| 5-FU alone VS no-chemotherapy | 0.979 | 0.99 (0.41, 2.38) |
| 5-FU + Platinum VS no-chemotherapy | 0.043 | 0.28 (0.08, 0.96) |
| 5-FU + others VS no-chemotherapy | 0.608 | 1.28 (0.50, 3.24) |
| Patient Group 4 | | |
| 5-FU alone VS no-chemotherapy | 0.491 | 0.73 (0.30, 1.79) |
| 5-FU + Platinum VS no-chemotherapy | 0.893 | 0.94 (0.37, 2.41) |
| 5-FU + others VS no-chemotherapy | 0.707 | 0.85 (0.36, 2.02) |

In Table 3, the hazard ratio (HR) was calculated using age, cancer stage, Lauren type, neural surrounding invasion state, and chemotherapy treatment as a regulator.

5. ACTA2 as Prognosis and Prediction Biomarker

It was further investigated whether among the 32 genes of the present disclosure, the expression of mRNA and protein of ACTA2 may be used to predict the overall survival rate of patients, chemotherapy, and immunotherapy responses.

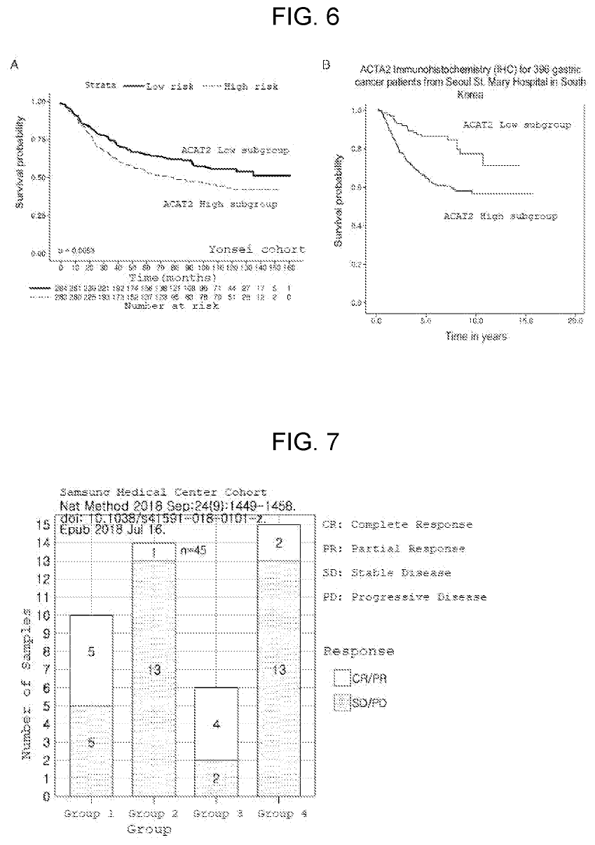

To this end, the patients from the Yonsei cohort were first divided on the basis of the average value of the expression of ACTA mRNA. Patients with higher ACTA2 mRNA expression showed a poor overall survival rate compared to patients with lower ACTA2 mRNA expression (FIG. 6A).

Multivariate Cox proportional analysis of age, tumor stage, tumor location, Lauren type, and ACTA2 mRNA expression associated with the risk of five-year death in cohorts of ACRG, Sohn et al. and the like, also showed that the 1-unit increase in ACTA2 mRNA expression was also associated with a higher risk with respect to the overall survival rate significantly and independently. The TCGA gastric cancer mRNA expression data also indicated that patient subgroups with high and low levels of ACTA2 showed statistically significant different overall survival outcomes.

To demonstrate the effectiveness of the prognosis of ACTA2 protein expression, the inventors of the present disclosure performed immunohistochemical analysis using an anti-ACTCA2 monoclonal antibody. Analysis of stained formalin-immobilized, paraffin embedded tissue sections from Seoul St. Mary Hospital (n=396) revealed the presence of subgroups of gastric cancer patients overexpressing ACTA2 protein in malignant epithelial and stromal cells. Patient subgroups with low ACTA2 protein expression showed better prognosis compared to patient subgroups with high ACTA2 protein expression (FIG. 6B).

ACTA2 Immunohistochemistry reading was performed according to the reading criteria of Table 4 below, read from tumor-surrounding stromal cells was performed in gastric cancer tissue microarray (TMA), and it was divided into two groups of Group 1 (ACTA2 low subgroup, score 0-3) and Group 2 (ACTA2 high subgroup, score 4-6) based on the score calculated by multiplying the staining intensity and the staining area score, respectively, and thus correlation with clinicopathologic factors and difference in survival rate of each group were analyzed.

TABLE 4

| | Intensity | Portion |
|---|---|---|
| 0 | No expression | <10% |
| 1 | Weak | 10-50% |
| 2 | Moderate | >50% |
| 3 | Strong | |

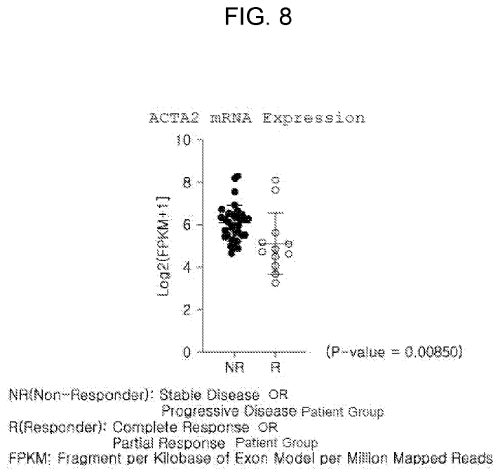

In addition, it could be confirmed that, as a result of measuring and analyzing the expression level of ACTA2 mRNA in the reaction group of patients who have received the immunotherapeutic agent treatment from Samsung Medical Center (N=45), it was confirmed that the patient subgroup resistant to the immunotherapeutic agent showed a higher expression of ACTA2 mRNA than the subgroup that responded to the immunotherapeutic agent (FIG. 8). In particular, it could be confirmed that a patient who does not react with the immunotherapeutic agent in the MSI-H patients exhibits high ACTA2 mRNA expression. In addition, patients who have reacted with the immunotherapeutic agent in the MSS patients were found to exhibit low ACTA2 mRNA expression.

That is, it could be confirmed that ACTA2 was overexpressed in a high-risk subgroup showing resistance to chemotherapy and immunotherapy.

6. Combination Evaluation of Biomarker and Microsatellite Instability High (MSI-H) Diagnosis of present disclosure

To examine the possibility of combining MSI diagnosis with prognosis prediction using the biomarker of the present invention, patients in the stomach cancer cohort of The Cancer Genome Atlas (TCGA) were divided into four subgroups based on MSI-H and MSS information and the mRNA expression level of ACTA2 as follows (FIG. 9).
1) MSI-H and ACTA2 High Subgroup
2) MSI-H and ACTA2 Low Subgroup
3) MSS and ACTA2 High Subgroup
4) MSS and ACTA2 Low Subgroup

In addition, to analyze that there is a statistically significant difference in survival rates between these MSI-H/MSS & ACTA2 high/low subgroups, a KM plot was created using the overall survival rate of patients in each subgroup (FIG. 10).

As a result, it was confirmed that there are a high expression level of ACTA2 mRNA and a small number of sub-groups in gastric cancer patients of MSI-H and MSS (FIG. 9), and there was a statistically significant difference in survival rate between the subgroups (FIG. 10).

In particular, it could be confirmed that the patients with the low expression level of ACTA2 mRNA (MSI-H or MSS+ACTA2 low) among MSI-H or MSS gastric cancer patients have a better prognosis than the subgroup of patients with MSI-H or MSS and ACTA2 high at the same time. Through this, it can be seen that the prognosis prediction of gastric cancer patients using existing MSI-H may be more accurately performed for the prognosis of gastric cancer patients through a combination of ACTA2 high or low biomarker.

In addition, in a method of selecting a gastric cancer patient sensitively responding to a chemical anticancer agent or an immunotherapeutic agent (or having poor prognosis) through the MSI-H biomarker combination, patients sensitive to chemotherapy and immunotherapy (e.g., MSI-H or MSS & ACTA2 low subgroups) and patients with resistance thereto (e.g. MSI-H or MSS & ACTA2 high subgroups) may be distinguished through ACTA2 biomarker combination.

Although the embodiments of the present disclosure have been described in detail above, the scope of the present disclosure is not limited thereto, and it would be obvious to a person skilled in the art that various modifications and variations are possible without departing from the technical spirit of the present disclosure as set forth in the claims.

1. A marker composition for predicting a prognosis of cancer, comprising:
an agent for measuring an expression level of mRNA or protein thereof of at least one gene selected from the group consisting of ESR1, BEST1, ACTA2, HIPK2, IGSF9, ASCC2, JUN, PPP2R5A, SMAD3, CREBBP, EP300 and DDX5.

2. The marker composition for predicting a prognosis of cancer of claim 1, wherein the marker composition for predicting the prognosis of cancer is for predicting a treatment prognosis of anticancer therapy, which is a survival rate, chemo-sensitivity, chemo-resistance, immunotherapy sensitivity, immunotherapy resistance, or any combination thereof.

3. The marker composition for predicting a prognosis of cancer of claim 1, wherein the composition comprises an agent for measuring an expression level of mRNA or protein thereof of an ACTA2 gene.

4. The marker composition for predicting a prognosis of cancer of claim 1, further comprising an agent for measuring an expression level of mRNA or protein thereof of at least one gene selected from the group consisting of FHL2, PML, BRCA1, WT1, AREG and TP63.

5. The marker composition for predicting a prognosis of cancer of claim 1, further comprising an agent for measuring an expression level of mRNA or protein thereof of at least one gene selected from the group consisting of TP53, HSF1, NCOA6IP, PAWR, FAM96A, WTAP, PCNA, GNL3, WRN, SMARCA4, NCOA6, RPA1, MSH6 and PARP1.

6. The marker composition for predicting a prognosis of cancer of claim 1, wherein the cancer is selected from the group consisting of breast cancer, stomach cancer, bladder cancer, kidney cancer, liver cancer, brain cancer, lung cancer, colon cancer, uterine cancer, skin cancer, and pancreatic cancer.

7. A method of predicting cancer prognosis, comprising:
measuring an expression level of mRNA or protein thereof of each gene of the marker composition for predicting a prognosis of cancer of claim 1; and
comparing the expression level of mRNA or protein thereof of the measured gene.

8. The method of predicting cancer prognosis of claim 7, wherein the prognosis is a survival rate, chemo-sensitivity, chemo-resistance, immunotherapy sensitivity, immunotherapy resistance, or any combination thereof.

9. The method of predicting cancer prognosis of claim 7, further comprising determining that a prognosis of chemotherapy is poor and a prognosis of immunochemotherapy is poor, when an expression level of mRNA or protein thereof of at least one gene selected from the group consisting of ESR1, BEST1, ACTA2, HIPK2, IGSF9, ASCC2, JUN, PPP2R5A, SMAD3, CREBBP, EP300 and DDX5 is relatively high.

10. The method of predicting cancer prognosis of claim 7, further comprising determining that prognosis of chemotherapy and immunotherapy is good when an expression level of mRNA or protein thereof of at least one gene selected from the group consisting of FHL2, PML, BRCA1, WT1, AREG and TP63 is relatively high.

11. The method of predicting cancer prognosis of claim 7, further comprising determining that a prognosis of chemotherapy is poor and a prognosis of immunotherapy is good when an expression level of mRNA or protein thereof of at least one gene selected from the group consisting of TP53, HSF1, NCOA6IP, PAWR, FAM96A, WTAP, PCNA, GNL3, WRN, SMARCA4, NCOA6, RPA1, MSH6 and PARP1 is relatively high, using the marker composition of claim 5.

12. A method of providing information for determining a treatment direction of cancer, the method comprising:
measuring an expression level of mRNA or protein thereof of at least one gene selected from a I gene group consisting of ESR1, BEST1, ACTA2, HIPK2, IGSF9, ASCC2, JUN, PPP2R5A, SMAD3, CREBBP, EP300 and DDX5, an expression level of mRNA or protein thereof of at least one gene selected from a II gene group consisting of FHL2, PML, BRCA1, WT1, AREG and TP63, and an expression level of mRNA or protein thereof of at least one gene selected from a III gene group consisting of TP53, HSF1, NCOA6IP, PAWR, FAM96A, WTAP, PCNA, GNL3, WRN, SMARCA4, NCOA6, RPA1, MSH6 and PARP1; and
by comparing the expression levels of mRNA or protein thereof of the measured gene, classifying as patient group 1 when the expression level of mRNA or protein thereof of the III gene group is relatively high among three gene groups, classifying as patient group 3 when the expression level of mRNA or protein thereof of the II gene group is relatively high, classifying as patient group 4 when the expression level of mRNA or protein thereof of the I gene group is relatively high, and classifying other patients as patient group 2.

13. The method of providing information for determining a treatment direction of cancer of claim 12, wherein in the patient group 2, the expression level of mRNA or protein thereof of the I to III gene groups is not distinguished between the I to III gene groups.

14. The method of providing information for determining a treatment direction of cancer of claim 12, wherein the method comprises at least one of predicting that the patient group 1 is inappropriate for anticancer therapy using chemical anticancer drug; predicting that the patient group 3 is suitable for anticancer therapy using chemical anticancer drug; and predicting that the patient group 4 is inappropriate for anticancer therapy using chemical anticancer drug.

15. The method of providing information for determining a treatment direction of cancer of claim 14, wherein the chemical anticancer drug is a complex anticancer drug in which at least one chemical anticancer agent selected from fluorouracil (5-FU), bleomycin, and epirubicin is combined based on platinum.

16. The method of providing information for determining a treatment direction of cancer of claim 12, wherein the method comprises at least one operation of predicting that at least one patient group of the patient group 1 and the patient group 3 is suitable for immunotherapy using an immunocancer agent; and predicting that at least one patient group of the patient group 2 and the patient group 4 is not suitable for immunotherapy using the immunocancer agent.

17. The method of providing information for determining a treatment direction of cancer of claim 16, wherein the immunocancer agent is at least one immunotherapeutic agent selected from an anti-PD1 inhibitor, an anti-CTLA4 inhibitor, and an anti-PDL1 inhibitor.

18. The method of providing information for determining a treatment direction of cancer of claim 12, further comprising diagnosing microsatellite instability (MSI) for determining a treatment direction of cancer.
